(19) United States
(12) Patent Application Publication
(10) Pub. No.: US 2023/0023847 A1
(43) Pub. Date: Jan. 26, 2023

(54) COMPOSITION FOR PREVENTING HAIR LOSS AND PROMOTING HAIR REGROWTH

(71) Applicant: LG HOUSEHOLD & HEALTH CARE LTD., Seoul (KR)

(72) Inventors: Yun-Ho CHOI, Seoul (KR); Jaeyoon KIM, Seoul (KR); Jae Young SHIN, Seoul (KR); Sanghwa LEE, Seoul (KR)

(73) Assignee: LG HOUSEHOLD & HEALTH CARE LTD., Seoul (KR)

(21) Appl. No.: 17/760,161

(22) PCT Filed: Feb. 5, 2020
(86) PCT No.: PCT/KR2020/001637

§ 371 Date: Aug. 4, 2022

(51) Int. Cl. A61K 8/60; A61Q 7/00

(52) U.S. Cl. CPC A61K 8/602; A61Q 7/00

(57) ABSTRACT

Provided is a method of alleviating or treating hair loss, or promoting hair growth by administering to a subject in need thereof composition comprising an active ingredient for preventing hair loss and inducing new hair growth or hair regrowth promotion by reducing the transition period from a telogen stage to an anagen stage in the hair growth cycle, and thereby exhibiting excellent effects of preventing hair loss and promoting hair regrowth by delaying conversion to a catagen stage.

### TECHNICAL FIELD

The present disclosure relates to a composition for preventing hair loss and promoting hair growth, which contains an ingredient for inducing hair growth or hair growth promotion.

### BACKGROUND ART

As the times change, interests in beauty are increasing day by day, in which hair is an important part. Hair is a keratinized structure produced from the skin surface. It serves as a cushion against external impact and protects the human body from external stimuli such as direct sunlight, cold, friction, etc. It is also responsible for excreting heavy metals such as arsenic, mercury, zinc, etc. out of the body and plays an important role in beauty, as the means of improving appearance. However, with the change in dietary habits or increased internal and external stresses, the number of people who suffer from hair loss due to many causes is increasing. Although genetic factors are known as the major cause of hair loss, increased social stress, environmental pollution, frequent perming and hair dyeing, wrong scalp management, etc. are also the causes of hair loss.

Human has about 100,000-150,000 hairs and each hair has a different growth cycle. The hair growth cycle consists of three stages: the anagen stage where hair grows the most actively, the catagen stage where the hair begins to degenerate, and the telogen stage where the hair growth stops or is in resting phase.

It is known that the activity of dermal papilla cells forming hair follicles and outer root sheath cells including hair follicle stem cells and various cytokines and growth factors produced by the cells play an important role in controlling the hair growth cycle. For example, Dickkopf-1 (DKK-1) plays an important role in the inhibition of growth and destruction of hair follicles (J Invest Dermatol, 2008: 128(2)). Meanwhile, the regeneration of hair follicles and activation of stem cells are necessary for cyclic hair growth. It is known that the regeneration and activation are achieved by the production and activation of Wnt/β-catenin (Nature 2007:447 (7142)).

In general, alopecia refers to a condition of abnormally increased depilation caused by decreased proportion of hair in the anagen stage and increased proportion of hair in the catagen or telogen stage in the hair growth cycle. In the case of a normal person, the proportion of hair in the anagen stage is high, while a person suffering from alopecia has a high proportion of hair in the telogen stage and shows visible hair loss. The persons suffering from alopecia are characterized by the downsizing of hair. As alopecia proceeds, the period of the anagen stage is reduced and the transition to the catagen and telogen stages is accelerated. Then, the volume of dermal papilla is reduced and hair follicles are downsized gradually. Therefore, in order to treat alopecia, it is important to recover hair in the telogen phase rapidly to the anagen phase and to extend the anagen stage.

Male-pattern hair loss, which is the alopecia with the most patients, is also called ‘androgenic alopecia’. Male-pattern hair loss occurs primarily in young men in their twenties to thirties. It is because androgenic alopecia is affected not only by genetic factors but also by the action of male hormone and age. The male hormone testosterone is known as the major cause of male-pattern hair loss, and the male-pattern hair loss is classified variously depending on the shape and progress of bald patches. Testosterone-induced male-pattern hair loss occurs as dihydrotestosterone (DHT) produced from the male hormone by the enzyme 5α-reductase acts on hair follicles to inhibit cell division and hair growth.

The type of hair loss changes depending on the distribution of the enzyme 5α-reductase on the scalp. Hair thinning and loss occur at the crown of the scalp where the enzyme is distributed a lot because cell division is decreased and the hair cycle is shortened due to DHT. In contrast, at the temporal or occipital part, hair loss does not occur easily because of relatively weak activity of the enzyme and the effect of female hormone. At present, hair transplantation, medication, etc. are used for management of male-pattern hair loss.

Although excessive action of male hormones, excessive sebum secretion, poor blood circulation, deterioration of scalp function due to peroxides, bacteria, etc., genetic factors, aging, stress, etc. have been discussed as the cause of hair loss, the cause of hair loss has not been clearly elucidated yet. Although currently available products for preventing hair loss contain ingredients for promoting blood circulation, enhancing hair root function, moisturizing the scalp, preventing dandruffs, preventing oxidation, extending the anagen phase, inhibiting the action of male hormone, etc. as active ingredients, they do not provide distinct effects and have side effects.

The medications for treating or preventing alopecia developed thus far include drugs containing female hormones as active ingredients, for promoting blood circulation, enhancing hair root function, moisturizing the scalp and inhibiting male hormones and drugs including minoxidil, finasteride, trichosaccharide. Minoxidil (2,4-diamino-6-piperidinopyrimidine 3-oxide), which is the representative topical medication for hair loss, increases blood flow to the scalp. And, finasteride, which is an oral medication used to treat hair loss, decreases the production of the active male hormone DHT by inhibiting the activity of 5α-reductase. However, these medications have many limitations due to side effects or cautions to be observed during use.

Meanwhile, gypsogenin 3-O-β-D-glucuronopyranoside, which is derived from baby's breath (Gypsophila paniculata Linn.), is known to inhibit the action of pancreatic lipase (Chemical and Pharmaceutical Bulletin. Vol 55, 2007, Issue 7).

In addition, deapioplatycodin D (deapi-platycodin D), which is derived from balloon flower (Platycodon grandijlorum (Jacq.) A. DC.), is known to be effective in inhibiting the proliferation of hepatitis virus (hepatitis C) (Evid Based Complement Alternat Med. 2013), reducing inflammation (Natural Product Sciences, 2008, 14(4): 274-80) and protecting the bronchus by promoting mucus secretin to the airway (Phytomedicine. 2014 Mar 15; 21(4)).

In addition, congmunoside X (congmuyanoside X, araloside X), which is derived from Chinese angelica-tree (Aralia chinensis L.) in the genus Aralia, is known to be effective in protecting neurons (Biomedicine & Pharmacotherapy, Vol 97, 2018 Jan, 152-161).

In addition, chikusetsusaponin IVa, which is derived from Japanese ginseng (Panax japonicus C. A. Mey.), is known to be effective in preventing obesity by regulating the differentiation of adipocytes (Nutrients 2018, 10(9), 1221), reducing inflammation (Int J of Immunopathology and Pharmacology 2015, Vol 28(3)) 308-317) and protecting heart cells (against hyperglycemia-induced myocardial injury) (Scientific Reports Volume 5, Article number, 2015, 18123).

However, the effect of gypsogenin 3-O-β-D-glucuronopyranoside, deapioplatycodin D, congmunoside X or chikusetsusaponin IVa related with the prevention of hair loss or promotion of hair growth has not been reported yet.

### Disclosure

### Technical Problem

The present disclosure is directed to providing a composition for preventing hair loss or promoting hair growth, which has superior effect of preventing hair loss or promoting hair growth while being safe for the human body with no side effect.

The present disclosure is also directed to providing a product for hair or scalp, which contains the composition for preventing hair loss or promoting hair growth.

### Technical Solution

The inventors of the present disclosure have made consistent efforts to develop a composition that can effectively promote hair growth while being safe for the human body without the problems of the side effects and cautions to be observed during use of the existing medications for treating hair loss. As a result, they have found out that a composition containing gypsogenin 3O-β-D-glucuronopyranoside, deapioplatycodin D, congmunoside X or chikusetsusaponin IVa as an active ingredient has a remarkably superior effect of preventing hair loss and promoting hair growth, and have completed the present disclosure.

Specifically, the inventors of the present disclosure have identified that each of the active ingredients described above is effective in inhibiting the activity of male hormones and inhibiting the expression of the hair follicle catagen factor Dkk-1. That is to say, it has been identified through in-vitro experiments that each of the active ingredients described above plays an important role in transiting the hair growth cycle from the catagen phase to the growth phase by inhibiting the activity of male hormones and inhibiting the expression of the hair follicle catagen factor. In addition, the inventors of the present disclosure have identified that the treatment of a composition for treating hair loss, containing the active ingredients, to patient with alopecia provides a remarkably superior effect of improving hair thickness, density and elasticity. Furthermore, as a result of comparative experiment with the commercially available minoxidil, it has been identified that the composition for treating hair loss containing the active ingredients provides a remarkably superior effect of preventing hair loss and promoting hair growth as compared to minoxidil.

The present disclosure provides a composition for preventing hair loss or promoting hair growth, which contains one or more selected from a group consisting of gypsogenin 3-O-β-D-glucuronopyranoside, deapioplatycodin D, congmunoside X and chikusetsusaponin IVa as an active ingredient.

In an exemplary embodiment, the four active ingredients may be derived from plants. The gypsogenin 3-O-β-D-glucuronopyranoside is an ingredient derived from baby's breath (Gypsophila paniculata Linn.), the deapioplatycodin D is an ingredient derived from balloon flower (Platycodon grandijlorum (Jacq.) A. DC.), the congmunoside X is an ingredient derived from Chinese angelica-tree (Aralia chinensis L.) in the genus Aralia, and the chikusetsusaponin IVa is an ingredient derived from Japanese ginseng (Panax japonicas C. A. Mey.).

In an exemplary embodiment, the gypsogenin 3-O-β-D-glucuronopyranoside may be contained in an amount of 0.0001-50 wt %, specifically 0.001-10 wt %, based on the total weight of the composition. The deapioplatycodin D may be contained in an amount of 0.0001-50 wt %, specifically 0.001-10 wt %, based on the total weight of the composition. The congmunoside X may be contained in an amount of 0.0001-50 wt %, specifically 0.001-10 wt %, based on the total weight of the composition. The chikusetsusaponin IVa may be contained in an amount of 0.0001-50 wt %, specifically 0.001-10 wt %, based on the total weight of the composition.

When the content of each active ingredient is 0.00001-50 wt %, specifically 0.0001-0.1 wt %, based on the total weight of the composition, superior effect of preventing hair loss and promoting hair growth is achieved in in-vitro experiments or clinically. When the content of the active ingredient is less than 0.0001 wt % based on the total weight of the composition, the effect of promoting hair growth is insignificant. And, when it exceeds 50 wt %, formulation stability is unsatisfactory.

The present disclosure also provides a pharmaceutical composition, a quasi-drug composition, a cosmetic composition or a functional health food composition, which contains the composition for preventing hair loss or promoting hair growth. The composition of the present disclosure may be prepared into any formulation that can be commonly applied to skin. Specifically, it may be prepared into a formulation for external application to skin. The composition of the present disclosure may be prepared into a formulation that can be applied to skin, for example, a liquid, a cream, a paste, a solid, etc.

The one or more selected from a group consisting of gypsogenin 3O-β-D-glucuronopyranoside, deapioplatycodin D, congmunoside X and chikusetsusaponin IVa of the present disclosure may be contained in the composition or formulation as an agent for preventing hair loss or an agent for promoting hair growth.

In an exemplary embodiment, when the formulation of the present disclosure is a liquid, a solvent, a solubilizer, an emulsifier, etc. may be used as a carrier ingredient. For example, water, an alcohol, ethyl carbonate, ethyl acetate, benzyl alcohol, benzyl benzoate, propylene glycol, 1,3-butylene glycol, glycerol, an aliphatic ester, polyethylene glycol, a fatty acid ester of sorbitan, etc. may be used. The alcohol may be specifically a linear or branched C₂-C₄ monoalcohol. Particularly, ethanol or isopropanol may be used, although not being limited thereto.

In an exemplary embodiment, when the formulation of the present disclosure is a paste, a cream or a gel, an alcohol, an animal oil, a plant oil, a wax, paraffin, starch, tragacanth, a cellulose derivative, polyethylene glycol, silicone, bentonite, silica, talc, zinc oxide, etc. may be used as a carrier ingredient. Specifically, the alcohol may be isopropanol, although not being limited thereto.

In an exemplary embodiment, when the formulation of the present disclosure is a powder or a spray, lactose, talc, silica, aluminum hydroxide, calcium silicate, polyamide powder, etc. may be used as a carrier ingredient. Particularly, a spray formulation may further contain a propellant such as chlorofluorohydrocarbon, propane/butane, dimethyl ether, etc.

In an exemplary embodiment, the composition of the present disclosure may further contain, in addition to gypsogenin 3O-β-D-glucuronopyranoside, deapioplatycodin D, congmunoside X and chikusetsusaponin IVa, ingredients commonly used in formulations for external application to skin. For example, it may further contain one or more additive selected from a group consisting of water, a surfactant, a wetting agent, an alcohol, a chelating agent, a sterilizer, an antioxidant, an antiseptic, a colorant, a flavorant, etc.

The present disclosure also provides a product for hair or scalp, which contains the composition for preventing hair loss or promoting hair growth. The product for hair or scalp may be one selected from a group consisting of a hair restorer, a scalp clinic agent, a scalp scaling agent, a scalp massage agent, a scalp care agent, a cleanser, a shampoo, a tonic, a hair conditioner, a hair lotion, a gel, a pack, a cream, an essence, a powder, a spray, an oil, a soap, an ointment, a hair styling agent, a hair dye, a hair perm agent, etc., although not being limited thereto.

In an exemplary embodiment, the composition of the present disclosure for preventing hair loss or promoting hair growth may be prepared into a formulation of a hair tonic or a hair lotion. Specifically, the composition of the present disclosure for promoting hair growth may be used through transdermal administration, e.g., by directly applying or spraying onto skin.

The present disclosure also provides a functional health food composition containing the composition for preventing hair loss or promoting hair growth. The functional health food may be prepared by a method commonly used in the art, and ingredients and components commonly used in the art may be added during the preparation. The formulation of the functional health food is not limited and any formulation acknowledged as long as it is acknowledged as a functional health food. The functional health food of the present disclosure may be prepared into various formulations. Unlike general drugs, it is advantageous in that it has no side effect, etc. that may occur during long-term intake because it is prepared from food ingredients and has superior portability. Therefore, the functional health food of the present disclosure may be used as an adjuvant for enhancing the effect of preventing hair loss and promoting hair growth.

The present disclosure also provides a method for preventing(alleviating) hair loss or promoting hair growth, which includes a step of administering a composition for preventing hair loss or promoting hair growth, which contains one or more selected from a group consisting of gypsogenin 3O-β-D-glucuronopyranoside, deapioplatycodin D, congmunoside X and chikusetsusaponin IVa as an active ingredient, to a patient (subject).

The term “administration” used in the present disclosure may mean introduction of the composition of the present disclosure by any appropriate method. The composition of the present disclosure may be administered through any general administration route by which the composition can be delivered to a target tissue. The composition of the present disclosure may be administered transdermally or dermally, specifically transdermally, most specifically by topical application. The number of application of the composition of the present disclosure may be determined depending on prescription, necessity or purpose.

The administration amount of the composition of the present disclosure may be adjusted adequately depending on the induvial difference in age, legions, etc. or formulation. Specifically, an appropriate amount may be applied to the scalp for 1 week to several months, once or several times a day. In a test example of the present disclosure, when the composition for treating hair loss 1 was used for 6 months, 7 times a week, a superior effect of promoting hair growth was achieved. In particular, a formulation containing ethanol exhibited a better effect of preventing hair loss and promoting hair growth (Test Example 3). The ethanol contained in the formulation can facilitate the transdermal absorption of gypsogenin 3O-β-D-glucuronopyranoside, deapioplatycodin D, congmunoside X and chikusetsusaponin IVa used as active ingredients of the present disclosure.

Through this, it was confirmed that the composition for preventing hair loss and promoting hair growth of the present disclosure, which contains gypsogenin 3O-β-D-glucuronopyranoside, deapioplatycodin D, congmunoside X and chikusetsusaponin IVa as active ingredients, can be used very effectively for treatment of hair loss. It will be obvious to those having ordinary knowledge in the art that the examples of formulation such as the hair tonic or the hair lotion are provided as examples of the composition for preventing hair loss or promoting hair growth of the present disclosure and the scope of the present disclosure composition is not limited by them.

### Advantageous Effects

The composition for preventing hair loss and promoting hair growth of the present disclosure exhibits superior effect of preventing hair loss and promoting hair growth by reducing the period of telogen-to-anagen transition in the hair growth cycle and delaying transition to the catagen phase.

### DESCRIPTION OF DRAWINGS

FIG. 1 shows the inhibitory effect of gypsogenin 3O-β-D-glucuronopyranoside against the activity of male hormone.

FIG. 2 shows the inhibitory effect of deapioplatycodin D against the activity of male hormone.

FIG. 3 shows the inhibitory effect of congmunoside X against the activity of male hormone.

FIG. 4 shows the inhibitory effect of chikusetsusaponin IVa against the activity of male hormone.

### BEST MODE

Hereinafter, the present disclosure is described in more detail through examples, etc. However, the examples of the present disclosure may be changed into various forms and it should not be construed that the scope of the present disclosure is not limited by the following examples. The examples of the present disclosure are provided so that the present disclosure is more completely understood by those having ordinary knowledge in the art.

### Test Example 1: Androgenic Activity

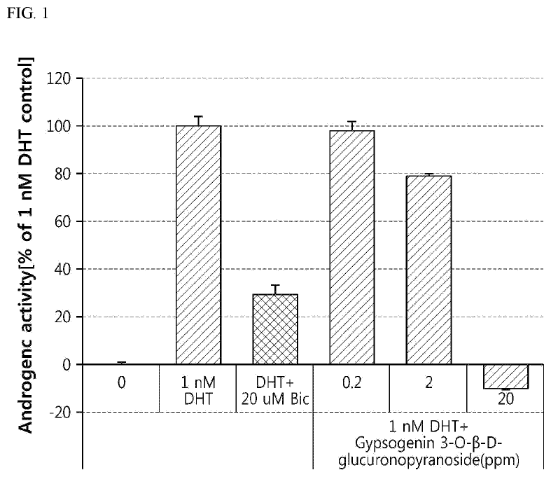

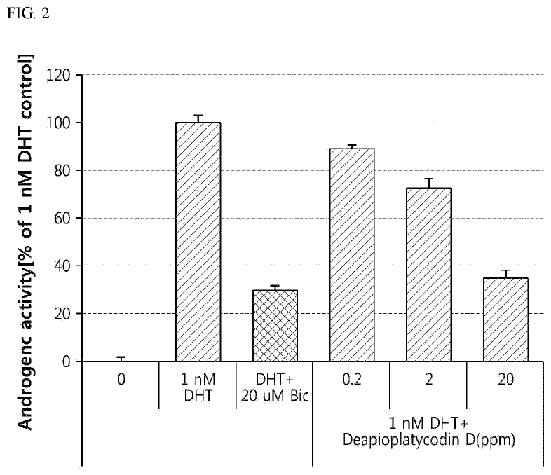

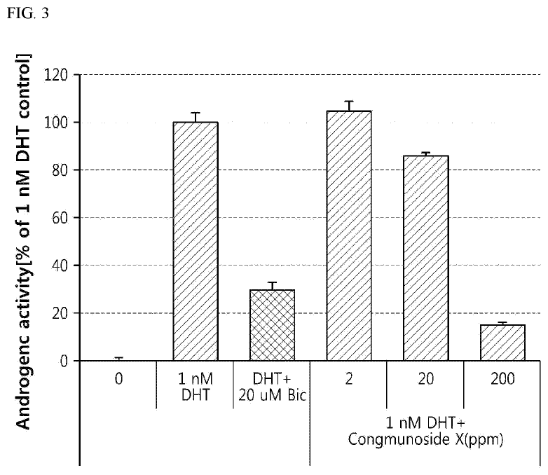

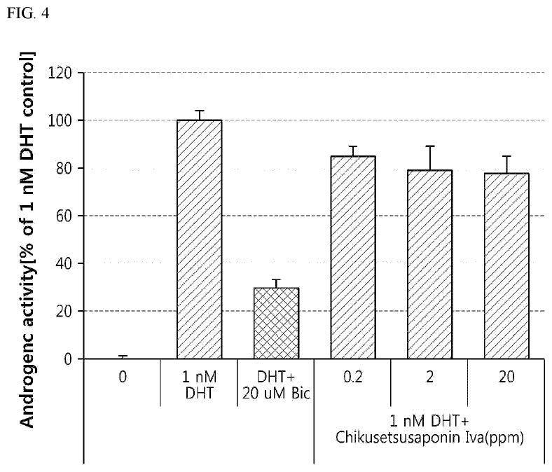

A stable cell line constructed by permanently transfecting androgen receptor-positive 22Rv1 human prostate cancer cells with the pGL4.36-MMTV-Luc vector, which possesses two androgen-responsive elements and a firefly luciferase reporter gene, was used for this experiment. The stable cell line was maintained by subculturing using RPMI1640 and 10% fetal bovine serum (GIBCO BRL, Gaithersburg, Md., USA). For transcriptional activation assay, the cells were seeded in a 96-well plate, with 25,000 cells per well, while replacing the medium with phenol red-free RPMI1640 containing 5% charcoal-stripped fetal bovine serum. After culturing in an incubator at 37° C. for 48 hours and then treating with gypsogenin 3O-β-D-glucuronopyranoside, deapioplatycodin D, congmunoside X or chikusetsusaponin IVa together with 1 nM DHT, the cells were cultured for 24 hours and the inhibition of luciferase activity increased by DHT was measured using a luciferase assay system (Promega). The result was expressed as the inhibition of the luciferase activity by gypsogenin 3O-β-D-glucuronopyranoside, deapioplatycodin D, congmunoside X or chikusetsusaponin IVa with respect to the luciferase activity increased by treatment with 1 M DHT as 100%. Bicalutamide (Casodex) was used as a positive control group and CCK-8 assay was conducted in parallel to investigate cytotoxicity. The cell culture was treated and incubated with CCK-8 at 1:10 for 2 hours. 2 hour later, the absorbance of each well was measured at 450 nm. All the experiments were 3 times and the absorbance was averaged. The result was expressed as percentage of the group treated with 1 nM DHT as 100%. The experimental result is shown in Table 1, Table 2 and FIGS. 1-4.

TABLE 1
Androgenic activity

| | | | | Androgenic |
|---|---|---|---|---|
| | | | | activity (% |
| Groups | | Concentration | | of 1 nM DHT) |
| Untreated group | | — | | 0 |
| DHT | | 1 | nM | 100.0 |
| 1 nM DHT | Bicalutamide | 20 | μM | 29.8 |
| | Gypsogenin 3-O-β-D- | 0.2 | ppm | 98.1 |
| | glucuronopyranoside | 2 | ppm | 79.1 |
| | | 20 | ppm | −9.8 |
| | Deapioplatycodin D | 0.2 | ppm | 89.1 |
| | | 2 | ppm | 72.2 |
| | | 20 | ppm | 34.9 |
| | Congmunoside X | 2 | ppm | 104.4 |
| | | 20 | ppm | 85.9 |
| | | 200 | ppm | 14.9 |
| | Chikusetsusaponin IVa | 0.2 | ppm | 84.7 |
| | | 2 | ppm | 79 |
| | | 20 | ppm | 77.5 |

TABLE 2
Cell viability

| | | | | Cell viability |
|---|---|---|---|---|
| Groups | | Concentration | | (% of 1 nM DHT) |
| Untreated group | | — | | 96.2 |
| DHT | | 1 | nM | 100.0 |
| 1 nM DHT | Bicalutamide | 20 | μM | 101.6 |
| | Gypsogenin 3-O-β-D- | 0.2 | ppm | 109.4 |
| | glucuronopyranoside | 2 | ppm | 107.6 |
| | | 20 | ppm | 104.8 |
| | Deapioplatycodin D | 0.2 | ppm | 102.2 |
| | | 2 | ppm | 99.4 |
| | | 20 | ppm | 81.6 |
| | Congmunoside X | 2 | ppm | 97.1 |
| | | 20 | ppm | 95.9 |
| | | 200 | ppm | 89.9 |
| | Chikusetsusaponin IVa | 0.2 | ppm | 102.0 |
| | | 2 | ppm | 105.2 |
| | | 20 | ppm | 99.4 |

### Test Example 2: Inhibition of Expression of Hair Follicle Catagen Factor

The inhibition of the expression of the hair follicle catagen marker Dkk-1 was investigated using human dermal papilla cells (hDPCs). The human dermal papilla cells were purchased from PromoCell. The hDPCs were subcultured in an hDPC culture medium (follicle dermal papilla cell growth medium; PromoCell, Heidelberg, Germany) containing a medium additive (Supplement Mix; PromoCell, Heidelberg, Germany), 100 units/mL penicillin and 100 ng/mL streptomycin under the condition of 37° C. and 5% CO₂. One million subcultured hDPCs were seeded onto a 100-mm cell culture dish and then cultured in an incubator at 37° C. 48 hours later, after treating with each of gypsogenin 3-O-β-D-glucuronopyranoside, deapioplatycodin D, congmunoside X and chikusetsusaponin IVa at 2 ppm or 20 ppm for 24 hours, the cell culture was subjected to Dkk-1 ELISA assay (R&D Systems, USA). The protein amount of each test group was identified by BCA assay and corrected with the result of Dkk-1 ELISA. The result is shown in Table 3.

TABLE 3
Expression of Dkk-1

| | Concentration | Relative expression level of |
|---|---|---|
| Groups | (ppm) | Dkk-1 (% of untreated group) |
| Untreated | — | 100.0 |
| Gypsogenin 3-O-β-D- | 2 | 96.5 |
| glucuronopyranoside | 20 | 77.5 |
| Deapioplatycodin D | 2 | 86.4 |
| | 20 | 76.7 |
| Congmunoside X | 2 | 90.8 |
| | 20 | 81.2 |
| Chikusetsusaponin IVa | 2 | 85.2 |
| | 20 | 70.9 |

### Test Example 3: Hair-Growing Effect of Composition for Treating Hair Loss 1

### (Hair Tonic)

Preparation of Composition for Treating Hair Loss 1 (Hair Tonic) Hair tonic compositions of Examples 1 to 4-2, which contain gypsogenin 3-O-β-D-glucuronopyranoside, deapioplatycodin D, congmunoside X or chikusetsusaponin Iva as an active ingredient, were prepared as described in Table 4.

TABLE 4

| | | | | | | | Flavorant | |
|---|---|---|---|---|---|---|---|---|
| | | | Castor | | | | and | Purified |
| Ingredients | | Ethanol | oil | Glycerin | Active ingredient | | colorant | water |
| Weight | Comp. | 55 | 5 | 3 | — | | Adequate | Balance |
| ratio (%) | Ex. 1 | | | | | | | (100 in |
| | Comp. | 55 | 5 | 3 | Minoxidil | 1 | Adequate | total) |
| | Ex. 2 | | | | | | | |
| | Ex. 1 | 55 | 5 | 3 | Gypsogenin 3-O-β-D- | 0.5 | Adequate | |
| | | | | | glucuronopyranoside | | | |
| | Ex. 1-2 | 0 | 5 | 3 | Gypsogenin 3-O-β-D- | 0.5 | Adequate | |
| | | | | | glucuronopyranoside | | | |
| | Ex. 2 | 55 | 5 | 3 | Deapioplatycodin D | 0.5 | Adequate | |
| | Ex. 2-2 | 0 | 5 | 3 | Deapioplatycodin D | 0.5 | Adequate | |
| | Ex. 3 | 55 | 5 | 3 | Congmunoside X | 0.5 | Adequate | |
| | Ex. 3-2 | 0 | 5 | 3 | Congmunoside X | 0.5 | Adequate | |
| | Ex. 4 | 55 | 5 | 3 | Chikusetsusaponin IVa | 0.5 | Adequate | |
| | Ex. 4-2 | 0 | 5 | 3 | Chikusetsusaponin IVa | 0.5 | Adequate | |

Preparation of Composition for Treating Hair Loss 2 (Hair Lotion)

Hair lotion compositions, which contain gypsogenin 3-O-β-D-glucuronopyranoside, deapioplatycodin D, congmunoside X or chikusetsusaponin IVa as an active ingredient, were prepared as described in Table 5.

TABLE 5

| Ingredients | Weight ratio (%) |
|---|---|
| Cetostearyl alcohol | 2 |
| Stearyl triethyl ammonium chloride | 2 |
| Hydroxyethyl cellulose | 0.5 |
| One or more of gypsogenin 3-O-β-D- | 0.01 |
| glucuronopyranoside, deapioplatycodin D, | |
| congmunoside X and chikusetsusaponin IVa | |
| Flavorant and colorant | Adequate |
| Purified water | Balance |
| | (100 in total) |

The compositions for treating hair loss 1 (hair tonic) of Comparative Examples 1 and 2 and Examples 1 to 4-2 were applied to the hair and scalp of a total of 110 males and females who have significantly fewer hairs than normal people or show the symptoms of alopecia and have weak hairs (10 groups with 11 people in each group) for 6 months, 7 times a week. Then, the improvement of hair thickness, hair density, hair elasticity and overall assessment (very good; +3, good; +2, slightly good; +1, no change; 0, slightly poor; −1, poor; −2, very poor; −3) was evaluated by the users 6 months later. The result is shown in Table 6.

TABLE 6

| | Hair | Hair | Hair | Overall |
|---|---|---|---|---|
| | thickness | density | elasticity | assessment |
| Comparative | 0.2 ± 0.3 | 0.1 ± 0.2 | 0.3 ± 0.4 | 0.2 ± 0.3 |
| Example 1 | | | | |
| Comparative | 2.2 ± 0.2 | 2.3 ± 0.4 | 2.1 ± 0.2 | 2.2 ± 0.3 |
| Example 2 | | | | |
| Example 1 | 3.6 ± 0.2 | 3.0 ± 0.2 | 3.3 ± 0.2 | 3.3 ± 0.2 |
| Example 1-2 | 2.6 ± 0.1 | 2.5 ± 0.3 | 3.0 ± 0.2 | 2.7 ± 0.2 |
| Example 2 | 3.1 ± 0.3 | 3.4 ± 0.1 | 3.1 ± 0.2 | 3.2 ± 0.1 |
| Example 2-2 | 2.8 ± 0.3 | 2.8 ± 0.2 | 2.5 ± 0.4 | 2.7 ± 0.3 |
| Example 3 | 2.9 ± 0.1 | 3.0 ± 0.2 | 2.7 ± 0.4 | 2.9 ± 0.2 |
| Example 3-2 | 2.5 ± 0.4 | 2.8 ± 0.3 | 2.5 ± 0.2 | 2.6 ± 0.3 |
| Example 4 | 2.9 ± 0.1 | 2.7 ± 0.3 | 2.8 ± 0.2 | 2.8 ± 0.2 |
| Example 4-2 | 2.2 ± 0.4 | 2.3 ± 0.3 | 2.4 ± 0.2 | 2.3 ± 0.3 |

1-9. (canceled)

10. A method of alleviating or treating hair loss, or promoting hair growth, comprising administering to a subject in need thereof, a composition comprising one or more active ingredient selected from the group consisting of gypsogenin 3-O-β-D-glucuronopyranoside, deapioplatycodin D, congmunoside X, and chikusetsusaponin Iva, and thereby alleviating or treating hair loss, or promoting hair growth in the subject.

11. The method of claim 10, wherein the active ingredient is in an amount of 0.0001-50 wt % based on the total weight of the composition.

12. The method of claim 10, wherein the composition inhibits the action of male hormones.

13. The method of claim 10, wherein the composition inhibits the expression of hair follicle catagen factor Dkk-1.
